(19) United States
(12) Patent Application Publication
(10) Pub. No.: US 2025/0072808 A1
(43) Pub. Date: Mar. 6, 2025

(54) WEARABLE DEVICE FOR DETECTING THE EYE MOVEMENT OF A USER

(71) Applicant: STMicroelectronics International N.V., Geneva (CH)

(72) Inventors: Enrico Rosario Alessi, Catania (IT); Daniele Baldacchino, Catania (IT); Fabio Passaniti, Siracusa (IT); Michele Alessio Dellutri, Catania (IT)

(21) Appl. No.: 18/804,589

(22) Filed: Aug. 14, 2024

(30) Foreign Application Priority Data
Aug. 28, 2023 (IT) 102023000017643

(51) Int. Cl. A61B 5/297; A61B 3/113; A61B 5/256

(52) U.S. Cl. CPC A61B 5/297; A61B 3/113; A61B 5/256

(57) ABSTRACT

A wearable device for detecting a user's eye movement having a first movement sensor with electrodes aligned with each other along a first movement axis, a second movement sensor with electrodes aligned with each other along a second movement axis transverse to the first movement axis, and a control circuit coupled to the electrodes and configured to acquire electrostatic charge variation signals indicative of differences between the electrostatic charge variations detected by the electrodes of the movement sensors; detect an event indicative of respective displacements of the eyes along the movement axis; and determine the movement of the eyes based on of the event confirmation.

### CROSS-REFERENCE TO RELATED APPLICATIONS

This application claims priority to Italian Application No. 102023000017643, filed on Aug. 28, 2023, which application is hereby incorporated by reference herein in its entirety.

### TECHNICAL FIELD

The present disclosure relates to a wearable device that detects a user's eye movement. Furthermore, it relates to an optical support apparatus that is wearable and includes the device, to a detection method for detecting the user's eye movement, and to a corresponding computer program product.

### BACKGROUND

As known, different current applications require monitoring the activity and, in more detail, the eye movements of the people. For example, eye-tracking is useful in applications based on smart glasses, where knowing the direction of the gaze of the person wearing them and acquiring commands provided by the person (e.g., voluntary blink, etc.) are important. For example, eye-tracking may activate advanced functions for adjusting the focal point of a camera lens or quick reading functions such as zoom and panoramic operations.

In general, eye-tracking requires monitoring the eye movements of the person to associate these movements with respective controls/functionalities and, therefore, allows the person to control electronic apparatuses without using gestures or voice commands. For example, eye movements may be grouped based on the following eye positions, considered in the person's eye reference system: center, top, bottom, right, left, top-left, top-right, bottom-left, and bottom-right.

Solutions are known that allow eye movements to be monitored. For example, electrooculography (EOG) is a technique to measure the cornea-retinal potential (CRP) that exists between the front portion (e.g., including the cornea) and the back portion (e.g., including the retina) of the human eye. The eye acts as an electric dipole where the front pole at the cornea is positive and the back pole at the retina is negative.

EOG is a commonly used measure as CRP is higher in intensity than electrical potentials generally monitored in other detection techniques (e.g., the electrical potentials involved in electroencephalography, i.e., EEG signals) since the eye is placed outside the skull and therefore no bone structure attenuates the generated electrical signal thereof. In EOG, to measure eye movement, a plurality of electrodes are placed around the eye and in contact with the facial skin; for example, five electrodes are distributed around the eye and on the forehead.

In greater detail, when the eye rotates around its own rotation center (e.g., substantially central to the same eyeball and to the eye socket), it generates a CRP variation detected through the electrodes. In embodiments, when the eyelid closes, the cornea moves in one direction, while when the eyelid opens, the cornea moves in the opposite direction, generating a CRP variation that is recognizable and associable with this blink. The signal resulting from a blink generally has a low frequency, for example, between 1 Hz and about 13 Hz.

However, this known measurement technique suffers from the following issues: measurement noise due to alternating electric current at 50 Hz or 60 Hz, possibly present in the environment where the measurement is carried out; noise due to head and body movements during the measurement; artifacts in the measurement caused by the operation of electrical apparatuses present in proximity to the electrodes; measurement errors due to contraction of the facial or neck muscles, or to the slipping of the electrodes on the skin due to sweat and eyelid blink. In general, EOG is a complex measurement technique with reduced measurement sensitivity.

### SUMMARY

The present disclosure aims to provide a wearable device for detecting the eye movement of a user, an optical support apparatus that is wearable and including the device, a detection method for detecting the eye movement of the user, and a corresponding computer program product, which overcome the drawbacks of the prior art.

According to the present invention, a wearable device for detecting the eye movement of a user, an optical support apparatus that is wearable and including the device, a detection method for detecting the eye movement of the user, and a corresponding computer program product, as defined in the annexed claims.

### BRIEF DESCRIPTION OF THE DRAWINGS

For a better understanding of the present invention, a preferred embodiment is now described, purely by way of non-limiting example, with reference to the attached drawings, wherein:

FIG. 1 is a block diagram that schematically shows a device for detecting the eye movements of a user, according to one embodiment;

FIG. 2 schematically shows the user wearing the device of FIG. 1, according to one embodiment;

FIGS. 3 and 4 show a pair of glasses, including the device of FIG. 1, according to respective embodiments;

FIG. 5 schematically shows three examples of possible positions of the user's eye with respect to the electrodes of the device in FIG. 1;

FIG. 6 schematically shows a visual system of the user;

FIG. 7 shows a detail of the visual scheme of FIG. 6;

FIGS. 8A-8D show respective plots exemplarily illustrating the trend over time of electrical quantities detected through the movement sensors of the device of FIG. 1, according to respective cases;

FIG. 9 is a block diagram that schematically shows a detection method performed through the device of FIG. 1, according to one embodiment;

FIGS. 10-11 show respective and further embodiments of the device of FIG. 1.

FIG. 12 is a flow chart of an embodiment method for detecting eye movement;

FIG. 13 is a block diagram of an embodiment device;

FIG. 14 is a flow chart of and embodiment detection method, which may be implemented in the device of FIG. 13; and

FIG. 15 is a flow chart of an embodiment detection method, which may be implemented in the device of FIG. 13.

In embodiments, the figures are shown with reference to a triaxial Cartesian system defined by an X-axis, a Y-axis, and a Z-axis, which are orthogonal to each other.

The following description indicates elements common to the different embodiments with the same reference numbers.

### DETAILED DESCRIPTION OF ILLUSTRATIVE EMBODIMENTS

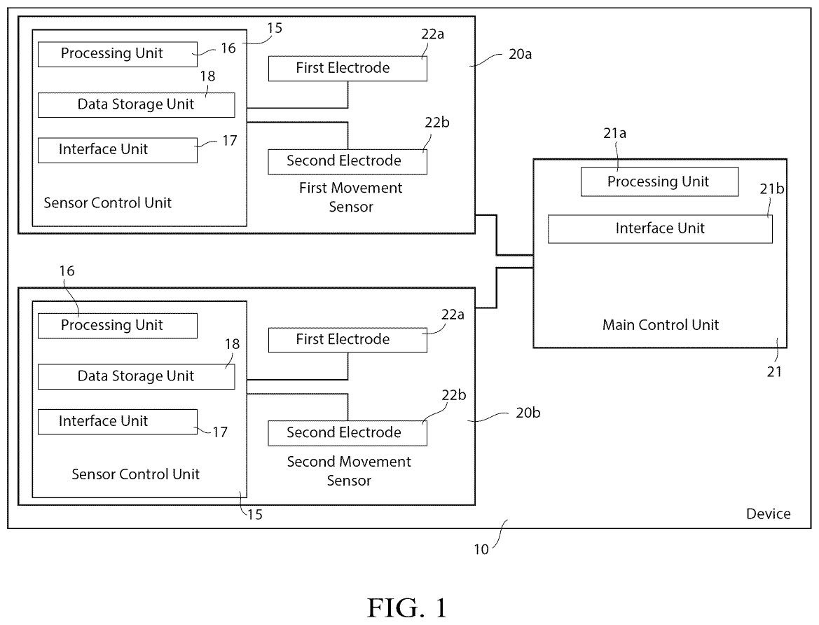

FIG. 1 shows a device 10 for detecting the activity of a person's eyes, particularly one or more eye movements.

As further described below, device 10 allows eye-tracking to be performed and may be used to control the functionality of an electronic apparatus (e.g., a screen, a TV, a PC) external to device 10 and operationally coupled thereto. As further described below, device 10 is wearable by a person (hereinafter also referred to as the user) in such a way as to allow the movements of his/her eyes to be detected. As shown in FIG. 1, device 10 includes a main control circuit 21, a first movement sensor 20a, and a second movement sensor 20b.

According to an exemplary embodiment, the main control circuit 21 (such as a microprocessor, a microcontroller, or a dedicated calculation circuit) is an electronic control circuit that may include, coupled to each other, a data storage circuit 21b (such as a memory, e.g. a non-volatile memory) for storing the acquired data and a processing circuit 21a for processing the acquired data.

For example, the processing circuit 21a may include a feature extraction module and a classifier module (e.g., “decision tree” or “support vector machine”). In a manner not shown and known per se, the main control circuit 21 may also include one or more of the following components: an electrical energy storage module (e.g., a battery), a power management module for electrical energy management, an interface analog front-end module with the first movement sensor 20a and the second movement sensor 20b, or a communication module (e.g., radio communication based on Bluetooth technology).

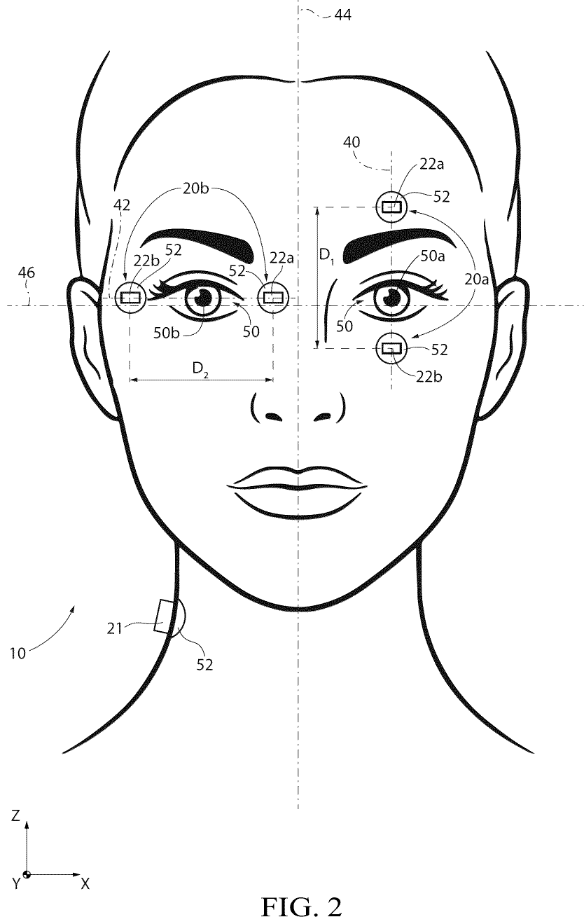

The first movement sensor 20a and the second movement sensor 20b are also referred to hereinafter as the first movement sensor 20a and the second movement sensor 20b. In embodiments, the first movement sensor 20a and the second movement sensor 20b are coupled to the main control circuit 21. As further described below with reference to FIGS. 2-4, the first movement sensor 20a and the second movement sensor 20b are placed at the person's eyes to detect the cornea-retinal potential (CRP) of the human eye.

In embodiments, the first movement sensor 20a is configured to detect the user's eye movement along a first movement axis, and the second movement sensor 20b is configured to detect the user's eye movement along a second movement axis that is transverse (in embodiments, orthogonal) to the first movement axis.

Thereafter, the exemplary and non-limiting case wherein the user's eye movement along the first movement axis corresponds to a vertical movement of the user's gaze (i.e., to the eye movement from bottom to top and vice versa), while the eye movement of the user along the second movement axis corresponds to a horizontal movement of the user's gaze (i.e., to the eye movement from left to right and vice versa) is considered.

In other words, the first movement axis corresponds to the vertical axis of the user's eye, and the second movement axis corresponds to the horizontal axis of the user's eye. For example, FIG. 2 shows the first movement axis with reference 40 and the second movement axis with reference 42.

In embodiments, exemplarily considering a front view of the user's face as shown for example in FIG. 2, the first movement axis 40 is parallel to a longitudinal axis (or axis of symmetry) 44 of the user's face and the second movement axis 42 is parallel to, or coincident with, a transverse axis 46 of the user's face. For example, the longitudinal axis 44 traverses the forehead and chin of the user and is equidistant from the eyes 50 of the user and the transverse axis 46 is orthogonal to the longitudinal axis 44 and passes through both eyes 50 of the user.

In greater detail, each first movement sensor 20a and the second movement sensor 20b includes a sensor control circuit 15 and two or more respective electrodes that are electrically coupled to the sensor control circuit 15.

In the embodiment exemplarily considered in FIG. 1, a first and a second electrode shown with the respective references 22a and 22b for each first movement sensor 20a and the second movement sensor 20b are exemplarily considered; however, the number of electrodes for each first movement sensor 20a and the second movement sensor 20b may be greater (e.g., four electrodes, as discussed hereinbelow).

The first electrode 22a and the second electrode 22b of the first movement sensor 20a are spaced from each other along the first movement axis, and the first electrode 22a and the second electrode 22b of the second movement sensor 20b are spaced from each other along the second movement axis, as further described below.

In embodiments, the first movement sensor 20a and the second movement sensor 20b are respective electrostatic charge variation sensors capable of detecting the CRP of the human eye.

In use, each first electrode 22a and the second electrode 22b detects a respective electrostatic charge variation caused by the user's eye movements, as discussed below, and generates a respective detection signal S_{R} indicative of this electrostatic charge variation.

In detail, each first electrode 22a and the second electrode 22b may have a metal surface or be of a totally metal material coated with a dielectric material, or even have a metal surface placed under an external case of the device 10. In any case, during use, each first electrode 22a and the second electrode 22b is electrostatically coupled to the environment in which device 10 is present, in more detail to the user's eye, which is more proximate to the first electrode 22a and the second electrode 22b considered, to detect the induced electrostatic charge variation thereof.

Each first electrode 22a and second electrode 22b may be integrated into the external case of device 10 and, for example, may be formed by a conductive track formed on, or in, a wafer of semiconductor material included in the device 10. Alternatively, each first electrode 22a and the second electrode 22b may be a metal element present in the device 10. Optionally, when a possible use of device 10 in a humid environment (more specifically in water) is envisaged, each first electrode 22a and the second electrode 22b may be inserted inside a waterproof case or, in any case, may be shielded using one or more protective layers, to prevent direct contact of the first electrode 22a and second electrode 22b with water or humidity: in this case, the waterproof case or the one or more protective layers are of a material (e.g., dielectric or insulating material, such as plastic material) such as not to shield the electrostatic charge generated by the user's eye, which is to be acquired by the first electrode 22a and second electrode 22b. Other embodiments are possible, as apparent to the person skilled in the art, so the first electrode 22a and the second electrode 22b are electrostatically coupled to the user's eyes during use.

Furthermore, according to an exemplary embodiment, the sensor control circuit 15 (such as a microprocessor, a microcontroller or a dedicated calculation circuit) is an electronic control circuit that may include, coupled to each other: an interface circuit 17 (optional and of known type) electrically coupled to the first electrode 22a and the second electrode 22b to interface the latter with the sensor control circuit 15 (e.g., the interface circuit 17 includes an amplification circuit or an analog-to-digital converter, ADC, not shown); a respective processing circuit 16 for processing detection signals S_{R} acquired through the first electrode 22a and the second electrode 22b (and optionally processed through the interface circuit 17); and a respective data storage circuit 18 (such as memory, e.g. a non-volatile memory) for storing the acquired data. For example, the sensor control circuit 15 is integrated into the first movement sensor 20a and the second movement sensor 20b.

In detail, each sensor control circuit 15 is configured to process (in a manner known per se, for example by amplifying and converting to digital) the respective detection signals S_{R} acquired through the first electrode 22a and second electrode 22b and to generate a respective electrostatic charge variation signal S_{Q,1}, S_{Q,2} indicative of a difference between the detection signals S_{R} acquired through the first electrode 22a and the second electrode 22b of the first movement sensor 20a and the second movement sensor 20b, respectively. In embodiments, the electrostatic charge variation signal S_{Q,1}, S_{Q,2} (e.g., of digital type) indicates a difference between the electrostatic charge variations detected through the first electrode 22a and the second electrode 22b. For example, S_{Q,1}=S_{R,1a}−S_{R,1b} (where S_{R,1a} is the detection signal S_{R} acquired through the first electrode 22a of the first movement sensor 20a and S_{R,1b} is the detection signal S_{R} acquired through the second electrode 22b of the first movement sensor 20a) and S_{Q,2}=S_{R,2a}−S_{R,2b} (where S_{R,2a} is the detection signal S_{R} acquired through the first electrode 22a of the second movement sensor 20b and S_{R,2b} is the detection signal S_{R} acquired through the second electrode 22b of the second movement sensor 20b).

FIG. 2 illustrates an embodiment of device 10 wherein the device 10 is directly worn by the user, i.e., wherein the first electrode 22a and the second electrode 22b of the first movement sensor 20a and the second movement sensor 20b are releasably coupled (in detail, removably bonded) to the user's facial skin.

In embodiments, in this embodiment device 10 may include a respective coupling element 52 for each first electrode 22a and second electrode 22b of the first movement sensor 20a and the second movement sensor 20b. The coupling elements 52 are fixed to the first electrode 22a and the second electrode 22b and allow the coupling (in detail, the temporary bonding) thereof to the facial skin of the user. In embodiments, the coupling elements 52 (e.g., patches, of known type) each have a respective first surface having the first electrode 22a and second electrode 22b fixed thereto and a second adhesive-type surface which, when placed in contact with the facial skin, adheres thereto and releasably couples the first electrode 22a and second electrode 22b to the facial skin of the user. For this purpose, the second adhesive surface has, for example, a layer of adhesive material (e.g., glue).

As shown in FIG. 2, the first electrode 22a and second electrode 22b of the first movement sensor 20a are aligned and spaced from each other along the first movement axis 40 and extend along the first movement axis 40 from sides opposite to each other of one of the two eyes 50 of the user (here exemplarily the left eye, or first eye, 50a of the user). Similarly, the first electrode 22a and second electrode 22b of the second movement sensor 20b are aligned and spaced from each other along the second movement axis 42 and extend along the second movement axis 42 from sides opposite to each of other of the two eyes 50 of the user (here exemplarily the right eye, or second eye, 50b of the user).

Although FIG. 2 shows the case wherein the first electrode 22a and second electrode 22b of the first movement sensor 20a are placed at the left eye 50a and the first electrode 22a and second electrode 22b of the second movement sensor 20b are placed at the right eye 50b, it is clear that this has been done for illustrative purposes and that other cases may be similarly considered, such as the case wherein the first electrode 22a and second electrode 22b of the first movement sensor 20a are placed at the right eye 50b and the first electrode 22a and second electrode 22b of the second movement sensor 20b are placed at the left eye 50a, the case wherein the first electrode 22a and second electrode 22b of the first movement sensor 20a and the second movement sensor 20b are all placed at the left eye 50a, or the case wherein the first electrode 22a and second electrode 22b of the first movement sensor 20a and the second movement sensor 20b are all placed at the right eye 50b.

For example, the first electrode 22a of the first movement sensor 20a may be placed on the upper eyelid of the left eye 50a (e.g., at the superior orbital septum), the second electrode 22b of the first movement sensor 20a may be placed under the lower eyelid of the left eye 50a (e.g., at the inferior orbital septum), the first electrode 22a of the second movement sensor 20b may be placed at the lacrimal caruncle of the right eye 50b and the second electrode 22b of the second movement sensor 20b may be placed at the end of the right eye 50b opposite to the lacrimal caruncle along the second movement axis 42.

For purely illustrative purposes, the first electrode 22a and second electrode 22b of the first movement sensor 20a are distant from each other along the first movement axis 40 by a first mutual distance D₁, for example, between about 5 cm and about 7 cm, and the first electrode 22a and second electrode 22b of the second movement sensor 20b are distant from each other along the second movement axis 42 by a second mutual distance D₂, for example, between about 6 cm and about 8 cm.

As shown in FIG. 2, the remaining components of device 10 are also wearable by the user, similarly to the first electrode 22a and second electrode 22b of the first movement sensor 20a and the second movement sensor 20b.

In embodiments, the main control circuit 21 may be fixed to a further coupling element 52, similar to those previously described and, for example, releasably bonded to the user's body (e.g., at the neck as exemplarily shown in FIG. 2).

Furthermore, and in a manner not shown in FIG. 2, each sensor control circuit 15 of the first movement sensor 20a and the second movement sensor 20b may be fixed to one of the coupling elements 52 of the first electrode 22a and second electrode 22b, may be fixed to the coupling element 52 of the main control circuit 21 or may be fixed to a further coupling element 52 releasably fixed to the user's body.

Furthermore, the main control circuit 21 is joined to the first movement sensor 20a and the second movement sensor 20b through electrical connections not shown, or electromagnetically (e.g., emitter module and receiver module included in the first movement sensor 20a and the second movement sensor 20b and in the main control circuit 21, respectively).

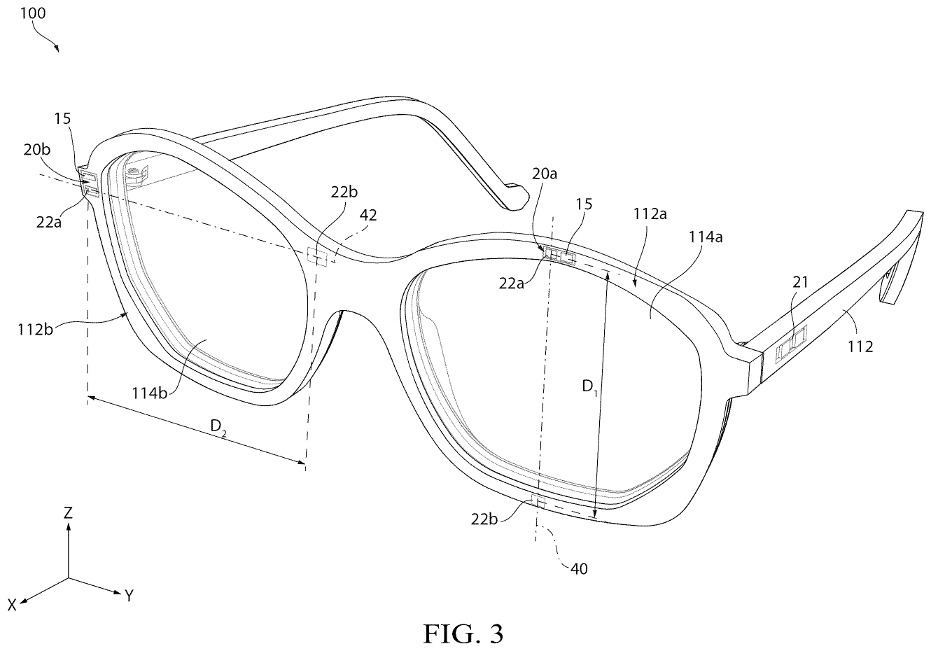

FIG. 3 illustrates an embodiment of device 10, where device 10 is included in an optical support apparatus 100 wearable on the user's face and configured to influence his vision.

In embodiments, the optical support apparatus 100 may be a pair of glasses or a headset (e.g., an artificial headset for gaming or augmented reality). For example, the glasses may be prescription glasses or sunglasses, or be of smart glasses type.

Thereafter, reference is made to the case wherein the optical support apparatus 100 is a pair of glasses. However, this is done only to simplify the description and not to limit it.

In embodiments, optical support apparatus 100 includes a frame 112 having a first support portion 112a and a second support portion 112b (e.g., of annular shape), which are configured to face the left eye 50a and the right eye 50b, respectively, when the user wears the optical support apparatus 100. In detail, the frame here may mean any support structure for glasses, headsets, etc., that face the user's eyes when in use.

In embodiments, the first support portion 112a and second support portion 112b support or accommodate a first lens 114a and a second lens 114b.

In the embodiment of FIG. 3, frame 112 carries the components of device 10, which are fixed thereto. For example, the main control circuit 21 is fixed to frame 112 and may be integrated therein.

The first movement sensor 20a and the second movement sensor 20b are carried by the first support portion 112a and the second support portion 112b of frame 112 and are placed in proximity to the user's eyes when the latter wears the optical support apparatus 100.

In embodiments, the first electrode 22a and second electrode 22b of the first movement sensor 20a are fixed to the first support portion 112a, are aligned and spaced from each other along the first movement axis 40 and extend along the first movement axis 40 from sides opposite to each other of the first lens 114a and therefore from sides opposite to each other of one of the two eyes 50 of the user when the optical support apparatus 100 are worn (here exemplarily the left eye 50a of the user). Similarly, the first electrode 22a and the second electrode 22b of the second movement sensor 20b are fixed to the second support portion 112b, are aligned and spaced from each other along the second movement axis 42, and extend along the second movement axis 42 from sides opposite to each other of the second lens 114b and therefore from sides opposite to each other of the two eyes 50 of the user (here exemplarily the right eye 50b of the user).

For example, the first electrode 22a and the second electrode 22b of the first movement sensor 20a and the second movement sensor 20b extend at the internal surfaces of the first support portion 112a and the second support portion 112b, which face the user's eyes when the latter wears the optical support apparatus 100.

For exemplary purposes, the first electrode 22a of the first movement sensor 20a may be carried by an upper portion of the first support portion 112a (e.g., proximate to the superior orbital septum), the second electrode 22b of the first movement sensor 20a may be carried by a lower portion of the first support portion 112a (e.g., proximate to the inferior orbital septum), opposite to the upper portion along the first movement axis 40, and the first electrode 22a of the second movement sensor 20b may be carried by a left portion of the second support portion 112b (e.g., at the bridge of the optical support apparatus 100 and therefore at the user's nose) and the second electrode 22b of the second movement sensor 20b may be carried by a right portion of the second support portion 112b (e.g., at the right arm of the optical support apparatus 100 and therefore on the opposite side of the second support portion 112b with respect to the bridge of the optical support apparatus 100), opposite to the left portion along the second movement axis 42.

Similarly to what has been previously described and for purely illustrative purposes, the first electrode 22a and second electrode 22b of the first movement sensor 20a may be distant from each other along the first movement axis 40 by the first mutual distance D₁, for example, between about 3 cm and about 5 cm, and the first electrode 22a and second electrode 22b of the second movement sensor 20b may be distant from each other along the second movement axis 42 by the second mutual distance D₂, for example, between about 6 cm and about 8 cm.

Furthermore, the main control circuit 21 is joined to the first movement sensor 20a and the second movement sensor 20b through electrical connections not shown, or electromagnetically (e.g., emitter module and receiver module included in the first movement sensor 20a and the second movement sensor 20b and in the main control circuit 21, respectively).

FIG. 4 shows a different embodiment of optical support apparatus 100, wherein the first electrode 22a and the second electrode 22b of the first movement sensor 20a and the second movement sensor 20b are not carried by frame 112 as previously described but are rather carried by the first lens 114a and second lens 114b. In embodiments first electrode 22a and second electrode 22b are integrated in the latter.

In embodiments, the first electrode 22a and the second electrode 22b of the first movement sensor 20a and the second movement sensor 20b of FIG. 4 are radially internal, with respect to the centers of the respective lenses 14a, 14b, to the first electrode 22a and the second electrode 22b of the first movement sensor 20a and the second movement sensor 20b of FIG. 3.

The first electrode 22a and the second electrode 22b of the first movement sensor 20a are fixed to the first lens 114a, are aligned and spaced from each other along the first movement axis 40 and may extend at respective ends of the first lens 114a which are opposite to each other along the first movement axis 40 (therefore they extend from sides opposite to each other of the left eye 50a, in the example considered here). Similarly, the first electrode 22a and second electrode 22b of the second movement sensor 20b are fixed to the second lens 114b, are aligned and spaced from each other along the second movement axis 42 and may extend at the respective ends of the second lens 114b which are opposite to each other along the second movement axis 42 (therefore they extend from sides opposite to each other of the right eye 50b, in the example considered here).

In detail, the first electrode 22a and the second electrode 22b of the first movement sensor 20a and the second movement sensor 20b may extend to the internal surfaces of the first lens 114a and second lens 114b, which face the user's eyes when the latter wears the optical support apparatus 100.

Consequently, the first electrode 22a and the second electrode 22b of the first movement sensor 20a may be distant from each other along the first movement axis 40 by a respective first mutual distance D₁′ smaller than the first mutual distance D₁ of FIGS. 2 and 3 and, for example, included about 2 cm and about 4 cm, and the first electrode 22a and the second electrode 22b of the second movement sensor 20b may be distant from each other along the second movement axis 42 by a respective second mutual distance D₂′ smaller than the second mutual distance D₂ of FIGS. 2 and 3 and, for example, between about 3 cm and about 5 cm.

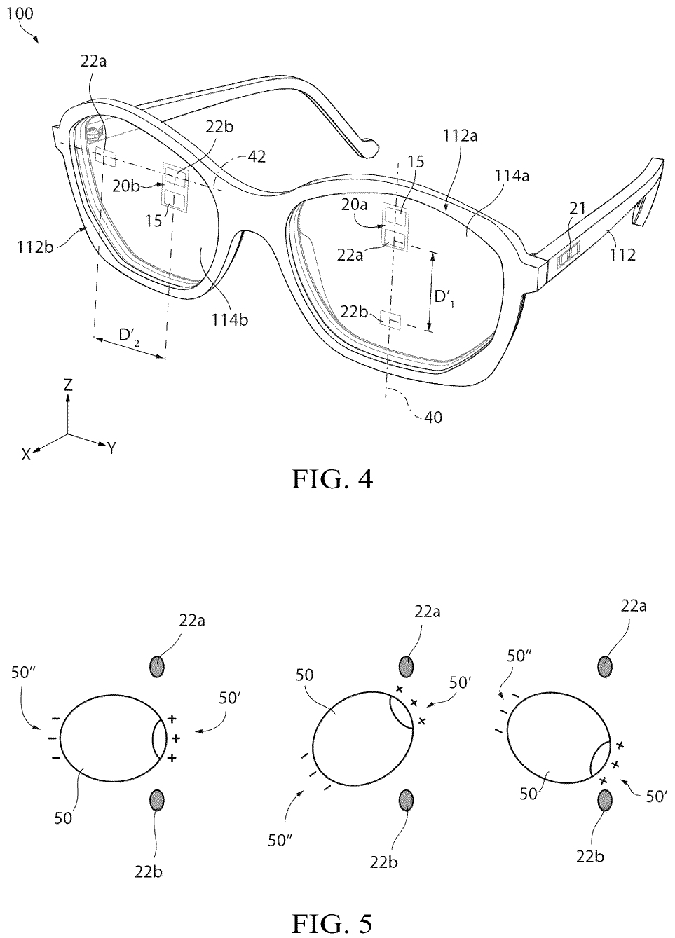

In general and as shown in FIG. 5, since eye 50 operates as an electric dipole with a positive pole at the cornea (indicated in FIG. 5 with the reference 50′) and a negative pole at the retina (indicated in FIG. 5 with the reference 50″), the movements of the eye 50 generate electric field variations in the environment surrounding the eye 50, and these electric field variations induce electrostatic charge variations which are detectable through the first electrode 22a and second electrode 22b. Since first electrode 22a and second electrode 22b are physically and electrically separated from each other, they are at different distances with respect to the cornea 50′ and the retina 50″ and, therefore, detect electrostatic charge variations different from each other. This allows for differential-type detection of electrostatic charge variations, as discussed below.

This allows both eye movements in the absence of blink (by the first movement sensor 20a or the second movement sensor 20b, as a function of the direction of movement of the eye 50) and the blinks of the user (by the first movement sensor 20a whose first electrode 22a and the second electrode 22b are aligned with each other parallel to the direction of movement of the eye 50 during the blink) to be detected. In fact, regarding the blink, it has been demonstrated that each blink corresponds to a respective electrostatic charge variation indicative of the blink.

In greater detail, it has been verified that such movements of the eyes 50 in the presence of blinks generate, in the corresponding electrostatic charge variation signal S_{Q,1} and in succession with each other in a blink period with a duration lower than about 50 ms, two respective peaks having sign opposite to each other with respect to a baseline of this signal. In embodiments, exemplarily considering a zero baseline, it is possible to have a first positive peak and a second negative peak or vice versa as a function of the direction of movement of the eyes 50 and the positions of the first electrode 22a and the second electrode 22b. These first and second peaks consecutive to each other in the blink period define a blink scheme (or pattern) indicative of a (voluntary or involuntary) blink. Greater details regarding the blink pattern and, in general, the detection of voluntary or involuntary blinks are provided in patent document EP4173567 of the same Applicant.

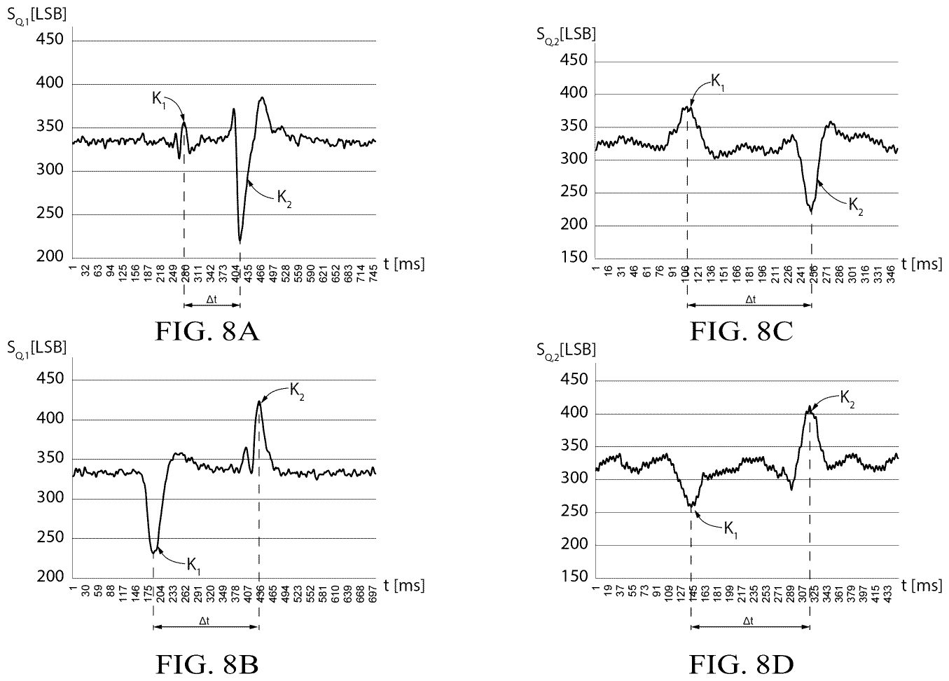

Instead, the movements of the eyes 50 in the absence of complete closure of the eyelids generate respective peaks, isolated from each other over time, in the electrostatic charge variation signals S_{Q,1} or S_{Q,2}. In embodiments, such peaks in the electrostatic charge variation signals S_{Q,1} or S_{Q,2} may be positive or negative (the latter are also referred to as valleys) with respect to respective baselines of the electrostatic charge variation signals S_{Q,1} or S_{Q,2}, as a function of the direction of movement of the eyes 50 and of the position of the first electrode 22a and second electrode 22b of the first movement sensor 20a and the second movement sensor 20b. Examples of such peaks in the electrostatic charge variation signals S_{Q,1}, S_{Q,2} are provided below in FIGS. 8A-8D.

In general, in the present discussion, a movement of the eye 50 is considered to be an angular displacement of the eye 50 around its rotation center and with respect to a predefined position that corresponds to a central position of the eye 50, i.e., to when the user looks straight ahead.

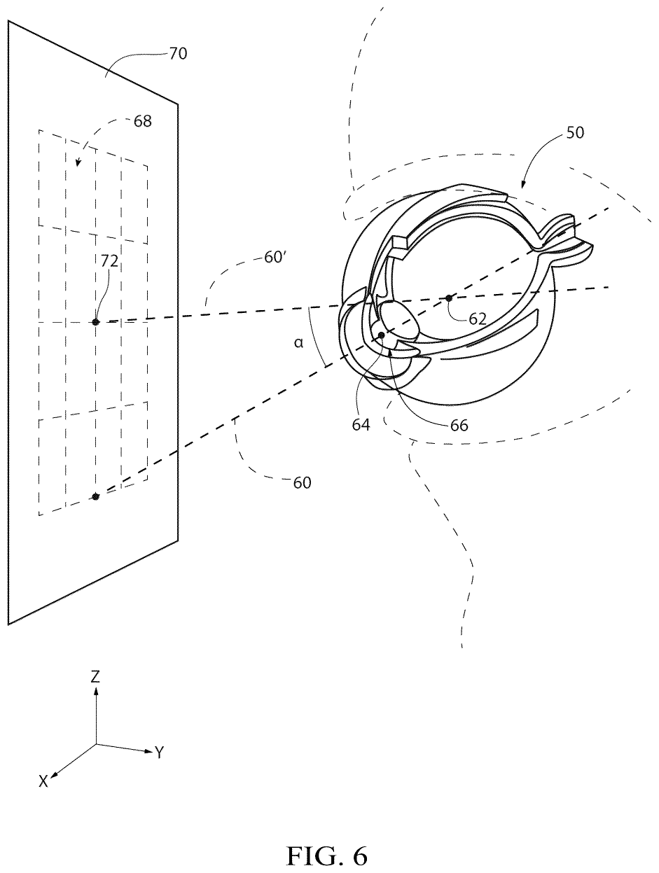

In greater detail and as shown in FIG. 6, a visual axis 60 is considered here along which the rotation center 62 of the eye 50 and a pupil center 64 of the pupil 66 of the eye 50 are aligned. In the central position of the eye 50, the visual axis 60 coincides with a reference axis 60′ which passes through the rotation center 62 and is substantially orthogonal to the user's face. When eye 50 rotates around its rotation center 62, the visual axis 60 deviates from the reference axis 60′ to be transverse thereto, and forms a rotation angle α (in detail, a solid angle defined in a reference system with polar coordinates with origin in the rotation center 62 of the eye 50) with the reference axis 60′. The angular displacement of the eye 50 identified by the rotation angle α corresponds to the movement of the eye 50 previously described.

In detail, the possible movements of the eye 50 may be classified and included in a plurality of predefined movement classes, each identified by a respective reference angular position of the eye 50. These reference angular positions are defined based on a visual grid 68 which lies on a reference plane 70 orthogonal to the reference axis 60′ and is centered in the intersection 72 of the reference axis 60′ with the reference plane 70.

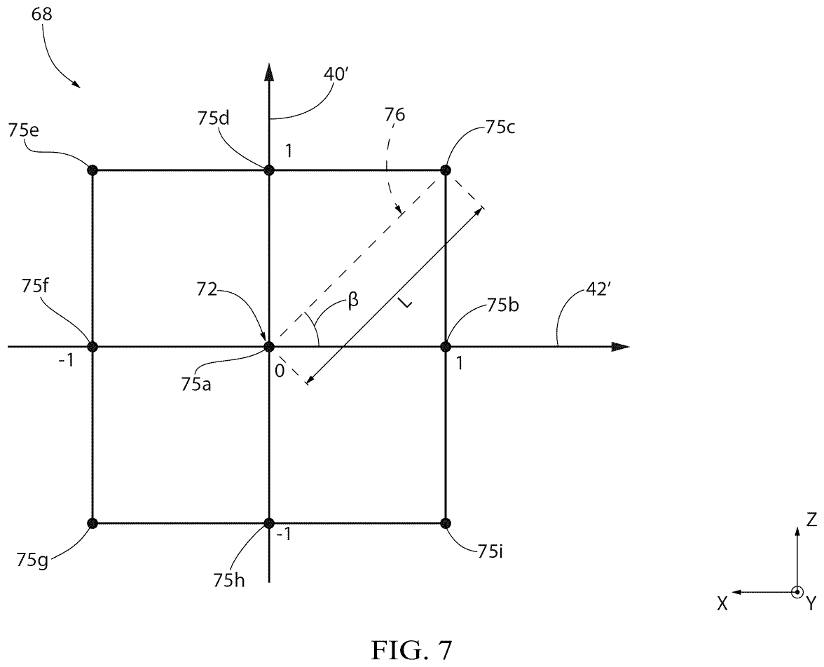

FIG. 7 shows an example of the visual grid 68, with a square shape. In embodiments, the visual grid 68 is shown in FIG. 7 in a biaxial Cartesian reference system with origin in the intersection 72 and defined by the grid axes 40′ and 42′, which are correlated to the first movement axis 40 and second movement axis 42, respectively. In detail, the grid axes 40′ and 42′ are parallel to the first movement axis 40 and second movement axis 42 respectively; for example, the grid axes 40′ and 42′ may respectively correspond to the projections of the first movement axis 40 and second movement axis 42 on the reference plane 70, or in any case are parallel to such projections.

In the visual grid 68 the following reference angular positions of the eye 50 are identified, defined in terms of normalized coordinates along the grid axes 40′: center 75a, coincident with the intersection 72 and defined by the coordinates (0,0); right position 75b, defined by the coordinates (1,0); top-right position 75c, defined by the coordinates (1,1); top position 75d, defined by the coordinates (0,1); top-left position 75e, defined by the coordinates (−1,1); left position 75f, defined by the coordinates (−1,0); bottom-left position 75g, defined by the coordinates (−1,−1); bottom position 75h, defined by the coordinates (0,−1); and bottom-right position 75i, defined by the coordinates (1,−1).

In embodiments, each reference angular position of the eye 50 (except for the center position 75a) may also be identified by a respective value of a tilting angle β defined between the positive semi-axis of the grid axis 42′ and an angular position axis 76 which joins the reference angular position considered and the center position 75a.

In detail, the right position 75b is defined by β=0°, the top-right position 75c is defined by β=45°, the top position 75d is defined by β=90°, the top-left position 75e is defined by β=135°, the left position 75f is defined by β=180°, the bottom-left position 75g is defined by β=225°, the bottom position 75h is defined by β=270°, the bottom-right position 75i is defined by β=315°.

These reference angular positions correspond to the respective positions of eye 50 in the user's visual reference system and, therefore, to the respective values of the rotation angle α. For example, the top-right position corresponds to when the user looks to the top-right, etc.

Each of these reference angular positions identifies a corresponding movement class of eye 50, i.e., a respective set of eye angular positions.

For purely illustrative and non-limiting purposes, the following movement classes of the eye 50 are defined as a function of the tilting angle β and the distance L of the considered angular position of the eye 50 from the intersection 72 (FIG. 7 exemplarily shows the distance L in the case of the top-right position 75c): a center class includes the sole center position 75a and therefore presents L=0 (in normalized value) and any β; a right class includes the right position 75b and is defined by L≠0 and 337.5°≤β<22.5°; a top-right class includes the top-right position 75c and is defined by L≠0 and 22.5° 0≤β<67.5°; a top class includes the top position 75d and is defined by L≠0 and 67.5°≤β<112.5°; a top-left class includes the top-left position 75e and is defined by L≠0 and 112.5°≤β<157.5°; a left class includes the left position 75f and is defined by L≠0 and 157.5°≤β<202.5°; a bottom-left class includes the bottom-left position 75g and is defined by L≠0 and 202.5°≤β<247.5°; a bottom class includes the bottom position 75h and is defined by L≠0 and 247.5°≤β<292.5°; a bottom-right class includes the bottom-right position 75i and is defined by L≠0 and 292.5°≤β<337.5°.

Nonetheless, other examples of definitions of the movement classes of the eye 50 may be similarly considered. For example, the movement classes may not be defined by angular endpoints that are angularly equi-spaced (i.e., they may be defined by angular ranges different from each other). According to another example of the definition of the movement classes of the eye 50, the same previously described ranges of the tilting angle β apply but the center class does not include the sole center position 75a and presents instead L≤Lₜₕ with Lₜₕ for example equal to 0.1, while the remaining movement classes present L>Lₜₕ.

FIGS. 8A-8D illustrate examples of electrostatic charge variation signals S_{Q,1} and S_{Q,2} acquired by the first movement sensor 20a and the second movement sensor 20b in different cases are now shown and discussed.

In embodiments, FIGS. 8A and 8B show examples of the first electrostatic charge variation signal S_{Q,1} acquired through the first electrode 22a and the second electrode 22b of the first movement sensor 20a. Consequently, the first electrostatic charge variation signal S_{Q,1} is indicative of movements of the left eye 50a along the first movement axis 40 (i.e. of the fact that the intersection of the visual axis 60 with the reference plane 70 displaces along the grid axis 40′).

In greater detail, FIG. 8A refers to the case wherein the user, starting from the center position 75a, first moves the eyes 50 downwards (positive main peak K₁, indicative of a downward movement) and then brings them back to the center position 75a (negative main peak K₂, indicative of an upward movement) after a time interval Δt (e.g., measured between the maximum amplitudes in absolute value of the main peaks K₁ and K₂). Instead, FIG. 8B refers to the opposite case wherein the user, starting from the center position 75a, first moves the eyes 50 upwards (negative main peak K₁, indicative of an upward movement) and then brings them back to the center position 75a (positive main peak K₂, indicative of a downward movement) after the time interval Δt.

FIGS. 8C and 8D instead show examples of the second electrostatic charge variation signal S_{Q,2} acquired through the first electrode 22a and second electrode 22b of the second movement sensor 20b. Consequently, the second electrostatic charge variation signal S_{Q,2} is indicative of movements of the right eye 50b along the second movement axis 42 (i.e., the intersection of the visual axis 60 with the reference plane 70 displaces along the grid axis 42′).

In greater detail, FIG. 8C refers to the case wherein the user, starting from the center position 75a, moves the eyes 50 first to the left (positive main peak K₁, indicative of a movement to the left) and then brings them back to the center position 75a (negative main peak K₂, indicative of a movement to the right) after the time interval Δt. Instead, FIG. 8D refers to the opposite case wherein the user, starting from the center position 75a, moves the eyes 50 first to the right (negative main peak K₁, indicative of a movement to the right) and then brings them back to the center position 75a (positive main peak K₂, indicative of a movement to the left) after the time interval Δt.

In use, the main control circuit 21 implements a detection method 150 to detect the movement of the eyes 50 of the user.

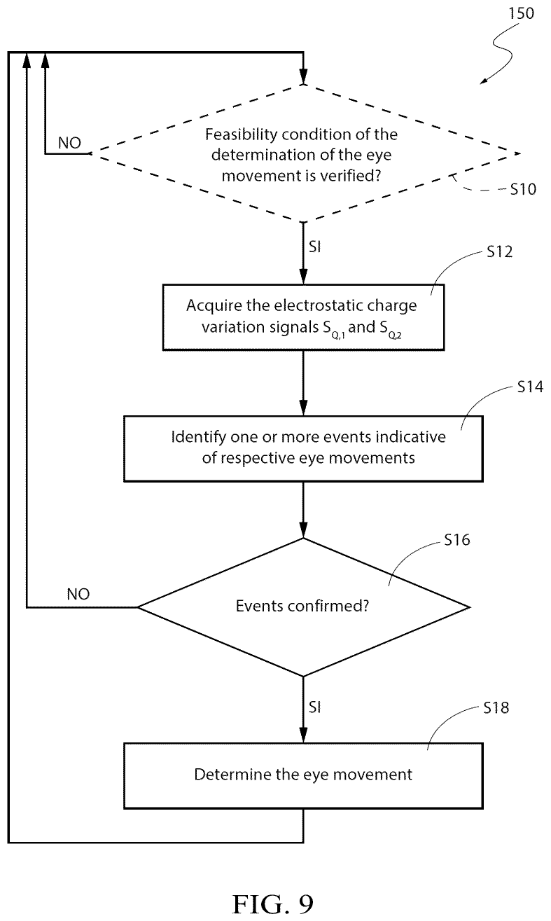

FIG. 9 exemplarily shows the detection method 150. In embodiments, an iteration of the detection method 150 is described below, but it is clear that the detection method 150 is of iterative type and, therefore, includes the execution of a plurality of these iterations in succession with each other.

In detail, at step S10 (optional) of the detection method 150, the main control circuit 21 verifies a feasibility condition of the determination of the movement of the eyes 50, i.e., it verifies whether or not the determination of the movement of the eyes 50 may occur in the time considered.

The feasibility condition may not be confirmed if one or more factors or events are detected that may affect the determination of the movements of the eyes 50, reducing its accuracy so much as to make the measurement not sufficiently informative potentially. For example, this may occur when the user moves his/her head. In this case, the direction of the user's gaze depends not only on the rotation of the eyes 50 in the eye sockets but also on the displacements of the head relative to a fixed and inertial reference system.

In embodiments, the verification of the feasibility condition here includes verifying whether the head movements of the user are present relative to a fixed and inertial reference system, for example, based on measurements performed through one or more inertial sensors (e.g., accelerometers or gyroscopes, In embodiments of MEMS type). In detail, these inertial sensors may be further included in the device 10, may be electrically or operatively coupled to the main control circuit 21, and may, for example, be carried by the frame 112 or be releasably coupled to the head of the user, similarly to what has been previously described.

In greater detail, the verification of the head movements occurs in a per se known manner, for example, by acquiring inertial data from one or more inertial sensors indicative of the occurrence or not of the user's head movements and determining the presence or absence of head movements based on this inertial data. This may occur in a per se known manner, for example, using machine learning techniques implemented through the feature extraction and classifier modules previously described.

For example, further details on how this verification might be performed can be found in the document “Using Inertial Sensors to Determine Head Motion-A Review,” Severin Ionut-Cristian and Dobrea Dan-Marius, Journal of Imaging, 2021.

If head movements are detected, the iteration stops, and the method does not proceed to the subsequent steps. In other words, in this case, the determination of the movement of the eyes 50 is not performed, as it might not be sufficiently accurate.

Alternatively, if no head movements are detected, the method proceeds to the steps subsequently described.

In step S10, it is also possible to determine the type of head movement or the user's activity, through per se known head-motion tracking and context-awareness techniques. In this case, it is possible to prevent the performance of subsequent steps when predefined head movements (e.g., very rapid or predefined-type movements) or predefined activities of the user (e.g., running, etc.) are more at risk of invalidating the subsequent measurement are detected, and to allow their performance in the opposite case.

At a step S12 consecutive to step S10, the main control circuit 21 acquires the first and the second electrostatic charge variation signals S_{Q,1}, S_{Q,2} respectively from the first movement sensor 20a and from the second movement sensor 20b. The first electrostatic charge variation signal S_{Q,1} is indicative of movements of the eyes 50 along the first movement axis 40 and the second electrostatic charge variation signal S_{Q,2} is indicative of movements of the eyes 50 along the second movement axis 42, transverse to the first movement axis 40. In other words, the movement of the eye 50 is here decomposed along two axes transverse to each other and is analyzed hereinbelow based on this decomposition.

At a step S14 consecutive to the step S12, the main control circuit 21 detects (i.e. identifies), starting from the electrostatic charge variation signals S_{Q,1} and S_{Q,2}, one or more possible events indicative of movements of the eyes 50 along the first movement axis 40 or the second movement axis 42, respectively.

In embodiments, this is done by verifying whether the electrostatic charge variation signals S_{Q,1} and S_{Q,2} present one or more peaks indicative of respective displacements of the eyes 50 along the first movement axis 40 and the second movement axis 42, respectively. These peaks are, for example, the main peaks K₁ and K₂ shown in FIGS. 8A-8D.

In greater detail, this may be done in the following manner.

Firstly, the electrostatic charge variation signals S_{Q,1} and S_{Q,2} may be filtered to remove the contributions induced by low-frequency alternating electric fields, possibly present in the environment surrounding the device 10. In fact, if any, an alternating electric field induces respective electric charge variations on the first movement sensor 20a and the second movement sensor 20b, generating respective peaks, in the frequency domain, in the electrostatic charge variation signals S_{Q,1}, S_{Q,2} at different frequencies (e.g., at the frequency of the alternating electric current, i.e. 50 Hz or 60 Hz depending on the Country one is in). In embodiments, the performed filtering may be of low-pass type with a cut-off frequency lower than a first threshold frequency (e.g., equal to about 25 Hz), or of band-pass type with a lower cut-off frequency greater than a second threshold frequency (lower than the first threshold frequency and for example equal to about 1 Hz) and with an higher cut-off frequency lower than the second threshold frequency and for example equal to 20 Hz, or of notch type with a lower cut-off frequency for example equal to 40 Hz, and with a higher cut-off frequency greater than about 70 Hz.

Furthermore, the electrostatic charge variation signals S_{Q,1} and S_{Q,2} may be processed to remove respective offsets of the latter, and in detail to subtract from each of them a respective baseline of the latter (i.e. a reference value, not necessarily constant in time and for example variable at very low frequency such as about 0.2 Hz, around which the respective electrostatic charge variation signal S_{Q,1} and S_{Q,2} develops; e.g., an average value). In this manner, hereinbelow, the variations of the electrostatic charge variation signals S_{Q,1} and S_{Q,2} concerning a zero baseline may be considered, and any offsets thereof are not to be considered.

Furthermore, the electrostatic charge variation signals S_{Q,1} and S_{Q,2} may be processed in a known manner to identify any peaks thereof (i.e., positive or negative peaks). In detail, it is possible to identify, if any, the number of peaks of the electrostatic charge variation signals S_{Q,1} and S_{Q,2} and, for each peak, a respective maximum value and a respective time position (or instant) of this maximum value. This is done in a per se known manner. For example, greater details regarding the identification modes of such peaks may be found in patent document US20220366768, of the present Applicant. Alternatively, the portions of the electrostatic charge variation signals S_{Q,1} and S_{Q,2} which have a greater value than a threshold value (e.g. a predefined value for example equal to about 200 LSB or a value equal to about 10% of the maximum value of the respective electrostatic charge variation signal S_{Q,1}, S_{Q,2} in the time window considered) may be considered as peaks.

Finally, the detected peaks of the electrostatic charge variation signals S_{Q,1}, S_{Q,2} may be validated or not based on reliability criteria, in order not to consider any peaks of the electrostatic charge variation signals S_{Q,1}, S_{Q,2} which are not indicative of movements of the eyes along the first movement axis 40 and second movement axis 42 and which for example may be caused by steps of the user or other people, sudden movements of the body on objects such as a plastic chair, etc.

In greater detail, the detected peaks of the electrostatic charge variation signals S_{Q,1}, S_{Q,2} meet the reliability criteria if they satisfy the conditions, for example, disclosed in patent documents of the same Applicant such as document US2023137601 (for example, with reference to the detection of involuntary blinks) and document US2022366768.

These so-far-described exemplary sub-steps of step S14 are provided for illustrative and non-limiting purposes and may be performed in succession with each other, or even just one subset thereof may be performed (e.g., the baseline is not removed).

If no events indicative of the movements of the eyes 50 along the first movement axis 40 or the second movement axis 42 have been detected (step S16 consecutive to step S14), the iteration ends, and the detection method 150 restarts from step S10.

Conversely, if events indicative of the movement of the eyes 50 along the first movement axis 40 or the second movement axis 42 have been detected (step S16 consecutive to step S14), the detection method 150 proceeds to step S18 consecutive to step S16.

At step S18, the main control circuit 21 determines the movement of the eyes 50 based on the events detected starting from the electrostatic charge variation signals S_{Q,1}, S_{Q,2}.

In embodiments, the eye movement is detected by reconstructing the biaxial movement as a function of the uniaxial displacements previously detected along at least one of the first movement axis 40 and second movement axis 42. In other words, the biaxial (i.e., overall) movement of the eyes 50 is generated starting from the mutual correlation of the uniaxial displacements (i.e., partial displacements, along a single axis) along the first movement axis 40 and second movement axis 42.

In case only one of the electrostatic charge variation signals S_{Q,1}, S_{Q,2} presents an event, it is determined that the detected movement of the eyes 50 occurs along only one of the two first movement axis 40 and second movement axis 42, i.e. the one corresponding to the electrostatic charge variation signal S_{Q,1}, S_{Q,2} which presents the event. For example, if the first electrostatic charge variation signal S_{Q,1} presents an event indicative of an upward movement along the first movement axis 40 and the second electrostatic charge variation signal S_{Q,2} presents no event, the movement of the eyes 50 has occurred only along the first movement axis 40 and is of top type.

In case both electrostatic charge variation signals S_{Q,1}, S_{Q,2} simultaneously present an event (i.e., present an event in respective time windows that are substantially superimposed on and coincident with each other), it is determined that the detected movement of the eyes 50 occurs along both the first movement axis 40 and second movement axis 42. For example, if the first electrostatic charge variation signal S_{Q,1} presents an event indicative of an upward movement along the first movement axis 40 and the second electrostatic charge variation signal S_{Q,2} presents an event indicative of a right movement along the second movement axis 42, the movement of the eyes 50 has occurred along the first movement axis 40 and second movement axis 42 and is of top-right type.

Furthermore, at step S18, the main control circuit 21 may also determine the new reference angular position of the eyes 50, i.e., update the reference angular position wherein the eyes 50 are located after the detected movement.

This can be done based on the visual grid 68 and the preceding reference angular position of the eyes 50.

For illustrative purposes, assuming that the preceding reference angular position was the center position 75a, detecting a top-right movement of the eyes 50 along the first movement axis 40 and second movement axis 42 implies that the reference angular position is updated to the top-right position 75c. The subsequent iteration will thus use this new reference angular position as a starting point to define the subsequent reference angular position.

Furthermore, and in a manner not shown, the detection method 150 may also include an optional step of controlling an electronic apparatus external to the device 10 based on the detected movement or the newly detected reference angular position. In embodiments, the electronic apparatus may be a TV, a screen, a PC, etc., and one functionality thereof may be controlled based on the detected movement or the new detected reference angular position (e.g., changing the television channel, modifying an option on the PC or tablet, etc.).

In embodiments, the method for identifying the eye's movement by elaborating the electrostatic charge variation signals S_{Q,1} and S_{Q,2} is based on artificial intelligence and machine learning (AI/ML) techniques.

The specific signals S_{Q,1} and S_{Q,2} provide a fingerprint of a specific eye movement. The signals can be filtered to remove the contributions induced by low-frequency alternating electric fields from powerline noise, and processed for removing the offset or baseline.

In embodiments, a dataset of distinctive patterns is built. The dataset can be formed by two vectors aligned on time linked to a specific eye movement where the first vector corresponds to, for example, the S_{Q,1} signal with K₁ and K₂ peaks for the left eye related to the horizontal movement. Further, the second vector corresponds to, for example, the S_{Q,2} signal with K₁ and K₂ peaks for the right eye related to the vertical movement. In embodiments, patterns are vectors of electrostatic charge variation signals, while the label corresponds to the specific eye movement that has generated the pattern itself.

When an eye activity is detected by processing the signals S_{Q,1} and S_{Q,2} in the simplest case by verifying that a signal variation over a pre-defined threshold is present, a pre-trained system can be used to classify the formed pattern in eye movements' classes, such as top-left, top or top-right; left, center, or right; bottom-left, bottom, or bottom-right; or a combination thereof.

In embodiments, the artificial intelligence or machine learning (AI/ML) system can be implemented by any of the well-known networks, such as a Convolutional Neural Network (CNN), a Temporal Convolutional Network (TCN), a Long Short-Term Memory (LSTM) network, or machine learning language models based on a learning phase of a labeled dataset.

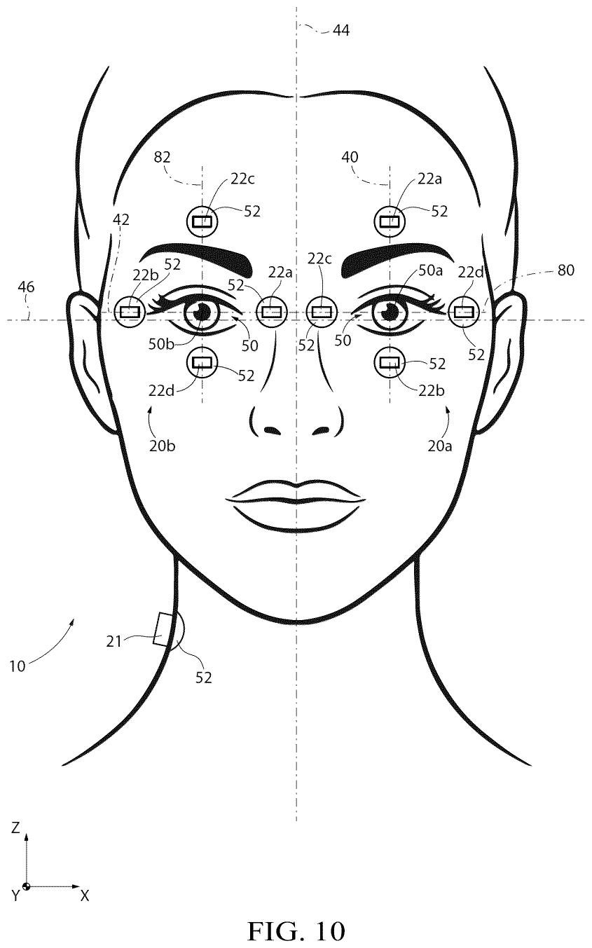

The method to identify the eyes' movement by elaborating the electrostatic charge variation signals S_{Q,1} and S_{Q,2} can be extended to using a plurality of electrodes on both the left and right eye. Thus, the distinctive pattern can be extended to the number of electrodes. In embodiments, the AI/ML system is pre-trained with the dataset formed by the extended patterns. If we consider the electrodes shown in FIG. 10, the pattern can form from four vectors as follows (1) SQ,1L from left eye electrodes (22a and 22b), (2) SQ,2L from left eye electrodes (22c and 22d), (3) SQ,1R from right eye electrodes (22a and 22b), and (4) SQ,2R from right eye electrodes (22c and 22d).

Generally, the advantages the disclosure affords can be shown by examining the characteristics of the invention made according to the present invention. The device 10 and the detection method 150 previously described allow, thanks to the proximity of the first electrode 22a and the second electrode 22b to the eyes 50, the movements of the eyes 50 to be detected with high accuracy.

Detecting eye movements 50 may be relevant in applications such as smart glasses that require knowing the direction in which the user is looking. For example, eye-tracking may activate advanced functions for adjusting the focal point of a camera lens or quick reading functions such as zoom and panoramic operations. Further applications regard detecting the user's attention level, the risk of falling asleep or simply activating functions in mobile and portable devices or smart TV-like applications.

Thanks to the inertial sensors, the possibility of performing the detection method 150 in the absence of the user's head movements avoids incorrect detections of eye movements, which is actually due to interferences generated by head movements.

Furthermore, in the embodiments of FIGS. 3 and 4, the fact that first electrode 22a and second electrode 22b are carried by frame 112 of the optical support apparatus 100 and are not in contact with the user's facial skin prevents detection errors due to slippage of the same on the skin (e.g., when the latter is moist or sweaty).

The detection method 150 requires reduced computational resources, minimizing the required electrical consumption.

Furthermore, it has been verified that the closer the first electrode 22a and the second electrode 22b is to the respective eye 50, the greater the performance in detecting the electrostatic charge variations induced by the movements of the eyes 50.

Furthermore, the differential measurement performed by each first movement sensor 20a and the second movement sensor 20b eliminates any common modes of the first electrode 22a and second electrode 22b measurements, improving the accuracy and robustness.

Finally, modifications and variations may be made to the invention described and illustrated herein without departing from the present invention's scope, as defined in the attached claims. For example, the described embodiments may be combined to provide further solutions.

Furthermore, device 10 may include a single control circuit coupled to first electrode 22a and second electrode 22b. For purely illustrative purposes, this control circuit may include the main control circuit 21 and the sensor control circuit 15 or it may be the main control circuit 21 and the sensor control circuit 15 may be absent; in these cases, the actions previously described concerning the sensor control circuits 15 of the first movement sensor 20a and the second movement sensor 20b are performed by the main control circuit 21.

Furthermore, concerning the embodiment of FIG. 2, the electrodes may be directly releasably fixed to the facial skin (i.e., without the need to use the coupling elements 52), for example, through pressure-coupling techniques. For example, dry electrodes (more details may be found at the link https://wearablesensing.com/dry-electrode/) may be used.

Furthermore, the number of electrodes of the first movement sensor 20a and the second movement sensor 20b may be greater than previously considered, as shown in FIGS. 10-12.

In embodiments, each first movement sensor 20a and the second movement sensor 20b may also include a third electrode 22c and a fourth electrode 22d, which are spaced both from each other and with respect to the first electrode 22a and the second electrode 22b of the first movement sensor 20a and the second movement sensor 20b and which are similar to the first electrode 22a and the second electrode 22b (and therefore are not described in detail again). The third electrode 22c and the fourth electrode 22d of each first movement sensor 20a and the second movement sensor 20b are also coupled to the sensor control circuit 15.

The third electrode 22c and the fourth electrode 22d of the first movement sensor 20a are aligned with each other along a first parallel movement axis 80 which is transverse (in detail, orthogonal) to the first movement axis 40 and, in embodiments, is parallel to the second movement axis 42 or coincident therewith. The third electrode 22c and the fourth electrode 22d of the first movement sensor 20a are arranged at the left eye 50a of the user, in a manner completely similar (and therefore not described again) to how the first electrode 22a and the second electrode 22b of the second movement sensor 20b are arranged relative to the right eye 50b.

In embodiments, the electrodes 22a-22d of the first movement sensor 20a are arranged as a cross or an X with respect to the left eye 50a. In other words, the intersection of the first movement axis 40 and the first parallel movement axis 80 occurs at the left eye 50a, for example at the respective pupil.

Similarly, the third electrode 22c and the fourth electrode 22d of the second movement sensor 20b are aligned with each other along a second parallel movement axis 82, which is transverse (in detail, orthogonal) to the second movement axis 42 and, in embodiments, is parallel to the first movement axis 40.

The third electrode 22c and the fourth electrode 22d of the second movement sensor 20b are arranged at the right eye 50b of the user, in a manner completely similar (and therefore not described again) to how the first electrode 22a and the second electrode 22b of the first movement sensor 20a are arranged relative to the left eye 50a.

In embodiments, the electrodes 22a-22d of the second movement sensor 20b are arranged as a cross or an X with respect to the right eye 50b. In other words, the intersection of the second movement axis 42 and the second parallel movement axis 82 occurs at the right eye 50b, for example at the respective pupil.

In this embodiment, each sensor control circuit 15 is also configured, similarly to what has been previously described for the first electrode 22a and the second electrode 22b, to process (in a per se known manner, for example by amplifying and converting into digital) the respective detection signals S_{R} acquired through the third electrode 22c and the fourth electrode 22d and to generate a respective electrostatic charge variation signal S_{Q,1}, S_{Q,2} indicative of a difference between the detection signals S_{R} acquired through the third electrode 22c and the fourth electrode 22d respectively of the first movement sensor 20a and the second movement sensor 20b. In embodiments, these electrostatic charge variation signals S_{Q,1}, S_{Q,2} are similar to what has been previously described and are each indicative of a respective difference between the electrostatic charge variations detected through the third electrode 22c and the fourth electrode 22d respectively of the first movement sensor 20a and the second movement sensor 20b.

For completeness of description, FIGS. 10-12 show the arrangement of the electrodes 22a-22d of the first movement sensor 20a and the second movement sensor 20b respectively according to the embodiments previously described with reference to FIGS. 2-4.

In general, the use of the third electrode 22c and the fourth electrode 22d for each first movement sensor 20a and the second movement sensor 20b makes the detection of the movements of the eyes 50 of the user more robust and reliable.

The measurements performed through the first electrode 22a and the second electrode 22b and the measurements performed through the third electrode 22c and the fourth electrode 22d may be used alternatively with each other in time, for example, in a periodic manner. For example, at the i-th iteration the detection method 150 uses the electrostatic charge variation signals S_{Q,1}, S_{Q,2} obtained through the first electrode 22a and the second electrode 22b of each of the first movement sensor 20a and the second movement sensor 20b and at the consecutive i+1-th iteration the detection method 150 uses the electrostatic charge variation signals S_{Q,1}, S_{Q,2} obtained through the third electrode 22c and the fourth electrode 22d of each of the first movement sensor 20a and the second movement sensor 20b.

This allows analyzing the movements of the eyes 50 in a completely symmetrical manner, for example, not to consider measurements relating to asymmetric gestures of the eyes (e.g., a voluntary blink of only one of the two eyes 50), or vice versa, to be able to take them into account and not mistake them for a symmetrical movement (e.g., an involuntary blink).

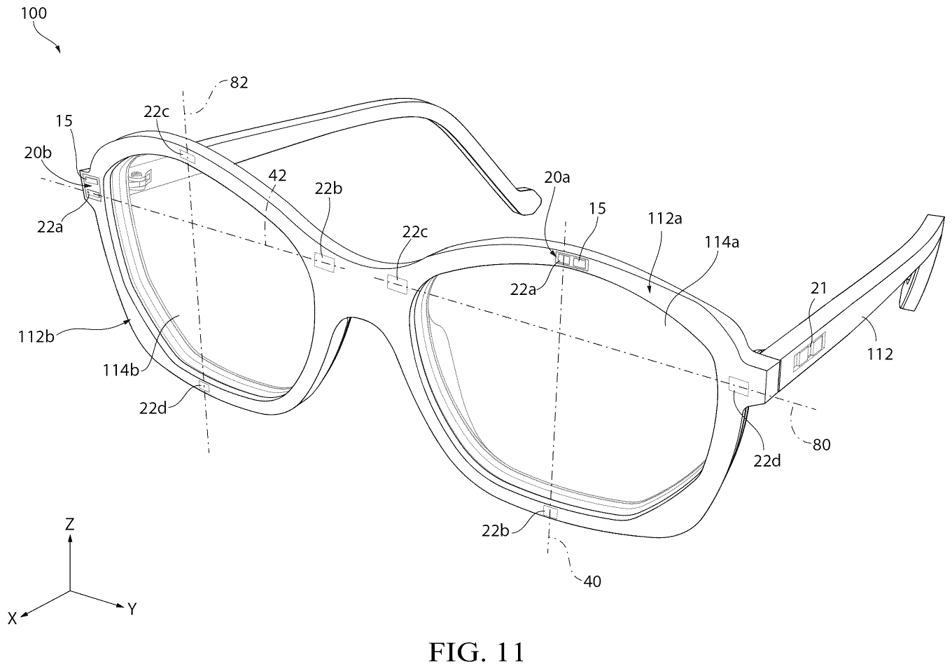

FIG. 12 illustrates a flow chart of an embodiment method 1200 for detecting eye movement.

At step 1202, raw data is collected from multiple electrodes. In embodiments, two separate sensor channels detect eye movement along different axes. For example, a first channel can be used to detect vertical eye movements, and a second channel can be used to detect horizontal eye movements. For example, in FIG. 11, the first channel may correspond to electrode 22a and the fourth electrode 22d of the second movement sensor 20b, and the second channel may correspond to the first electrode 22a and second electrode 22b of the first movement sensor 20a.

At step 1204, individual low-pass filtering is performed on the raw data for each channel. For example, the low-pass filter may have a cutoff frequency of approximately 2 Hz, chosen to capture the typical frequency range of human eye movements while excluding faster fluctuations that may represent noise or artifacts. In embodiments, individual low-pass filtering is performed on the raw data for horizontal and vertical eye movements. Filtering, advantageously, isolates relevant eye movement signals from higher-frequency noise. By applying the filter separately to the horizontal and vertical channels, each eye movement dimension is processed appropriately. The filtering step helps to smooth the raw data, removing rapid fluctuations and high-frequency interference that could otherwise lead to false detections or inaccuracies in subsequent processing stages.

At step 1206, individual adaptive baseline removal is done on the filtered data for each channel to eliminate any offset or drift. Once the raw data from the electrodes are filtered, offsets or drifts in the signal are eliminated using the adaptive baseline removal step. Individual adaptive baseline removal helps to isolate the actual eye movement signals from any background fluctuations or slow changes in the baseline voltage. Removing unwanted variations makes eye movement detection and measurement more accurate. The adaptive nature of the process allows the adjustment of changes in the baseline over time, which is advantageous to maintain accuracy during extended periods of use.

At step 1208, each channel's filtered and baseline-removed data are individually amplified to enhance signal strength. The amplification can improve the signal-to-noise ratio of the eye movement data, making subtle eye movements more detectable and easier to analyze. The amplification can involve applying a gain factor to the horizontal and vertical eye movement channels. By increasing the amplitude of the signals, genuine eye movements from remaining background noise or small fluctuations can be better distinguished.

At step 1210, a modulus (MOD) computation is performed using the amplified, filtered, and baseline-removed data. This step combines the horizontal and vertical components to determine the overall magnitude of eye movement. In embodiments, the modulus (MOD) signal (r) can be represented as r=\sqrt{(x²+y²)}, where x represents the horizontal component and y represents the vertical component of the data.

At step 1212, peak detection and width analysis are performed on the modulus signal to identify significant eye movements and their durations. In embodiments, peak detection includes locating local maxima in the modulus signal, corresponding to instances of substantial eye movement above a certain threshold. Thus, peak detection includes a first step where the modulus signal is determined to exceed a threshold value (r_{threshold}). For the period that the module signal exceeds the threshold value, the peak value within that period corresponds to the modulus signal peak (rₚₑₐₖ). Alongside peak detection, a width analysis is performed at step 1212, which involves measuring the duration or temporal extent to which the modulus signal exceeds the threshold value. The modulus signal exceeding the threshold value (r_{threshold}) indicates an eye event.

At step 1214, the method concludes by detecting the eye angle. In embodiments, the eye angle (θ) is calculated for the modulus signal peak, as determined in step 1212. In embodiments, the eye angle (θ) can be calculated based on the equation θ=arctan(yᵣₚₑₐₖ, Xᵣₚₑₐₖ), where yᵣₚₑₐₖ is the vertical component at the modulus signal peak (rₚₑₐₖ) and xᵣₚₑₐₖ is the horizontal component at the modulus signal peak. The eye angle (θ) determines whether the user is looking.

Advantageously, by converging the horizontal and vertical eye movement channel outputs through the modulus signal, method 1200 improves angle detection accuracy and is not limited to detecting whether the eye is directed towards the top, bottom, right, left, top-left, top-right, bottom-left, or bottom-right—it can provide an angle degree between 1 and 360 degrees.

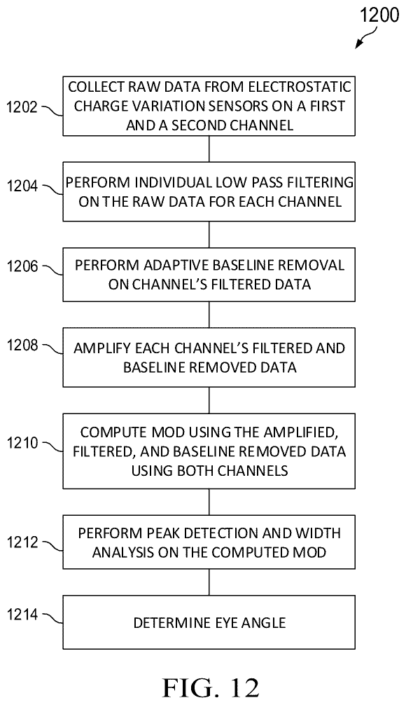

FIG. 13 illustrates a block diagram of an embodiment device 1300. Device 1300 includes the first movement sensor 20a, the second movement sensor 20b, the main control circuit 21, a memory, and a sensor 1304, which may (or may not) be arranged as shown. Device 1300 may include additional components not shown, such as various interfaces or other types of sensors. The details of components previously discussed are not repeated for the brevity of the discussion.

Device 1300 may be any device that can take advantage of embodiments disclosed to detect eye movement and determine the eye angle of the user wearing the device. For example, device 1300 may be a smart glass (commercial or consumer-based), an augmented reality (AR) headset, a virtual reality (VR) headset, a mixed reality (MR) device, a smart helmet, a medical diagnostic device, an automotive head-up (HUD) display, a gaming headset, or the like.

Memory 1302 may be any component or collection of components adapted to store programming, instructions, or calibration settings for execution or retrieval by the main control circuit 21. In an embodiment, memory 1302 includes a non-transitory computer-readable medium. In embodiments, memory 1302 is embedded within the main control circuit 21.

Sensor 1304 can be any component or collection of components for speech activity detection adapted to determine whether the user wearing the device 1300 is speaking. In embodiments, the accelerometer is positioned to detect vibrations associated with the user's head movements and speech.

In embodiments, sensor 1304 is an accelerometer configured to measure acceleration forces in various directions. In embodiments, it can measure data along the X, Y, and Z axes. The main control unit 21 can calculate the norm of the accelerometer data using equation (1): accelerometer norm=\sqrt{X²+Y²+Z²}. In embodiments, the sensor 1304 is a Micro-Electro-Mechanical System (MEMS) type of accelerometer. MEMS accelerometers are compact, have low power consumption, and have high precision. In embodiments, sensor 1304 may be coupled to an analog-to-digital converter (ADC) to convert the accelerometer sensor output to a digital signal.

In embodiments, sensor 1304 includes a microphone that can detect vocal vibrations and sound patterns associated with speech. In embodiments, sensor 1304 includes a bone conduction sensor that detects vibrations through the bones of the skull that occur during speech. In embodiments, sensor 1304 includes an electromyography (EMG) sensor placed near the temples or jaw that can detect muscle activity associated with speaking. In embodiments, sensor 1304 includes a proximity sensor near the mouth of the user that can detect changes in proximity when the jaw moves during speech. In embodiments, sensor 1304 includes an infrared sensor aimed at the throat or jaw that detects temperature changes or movement associated with speech. In embodiments, sensor 1304 includes an optical sensor that can detect subtle movements of the skin or changes in blood flow associated with facial muscle activity during speech. In embodiments, sensor 1304 includes a strain gauge sensor integrated into the frame of the device 1300 that can detect minute deformations caused by facial movements during speech. In embodiments, the sensor 1304 is integrated into the arms or bridge of glasses that make contact with the user's head.

In embodiments, the sensor 1304 is embedded within the first movement sensor 20a, the second movement sensor 20b, or both. For example, the movement sensors may include an accelerometer that can advantageously detect speech-related activities by the user. Therefore, in devices that already include an embedded sensor 1304, the device can determine speech activity without requiring or adding additional components.

In embodiments, the main control circuit 21 is configured to receive data from the sensor 1304 and, based thereon, determine whether the user is speaking. For example, if the sensor 1304 is an accelerometer, the main control circuit 21 can determine whether the user is speaking if the accelerator norm exceeds a threshold value or is within a threshold range.

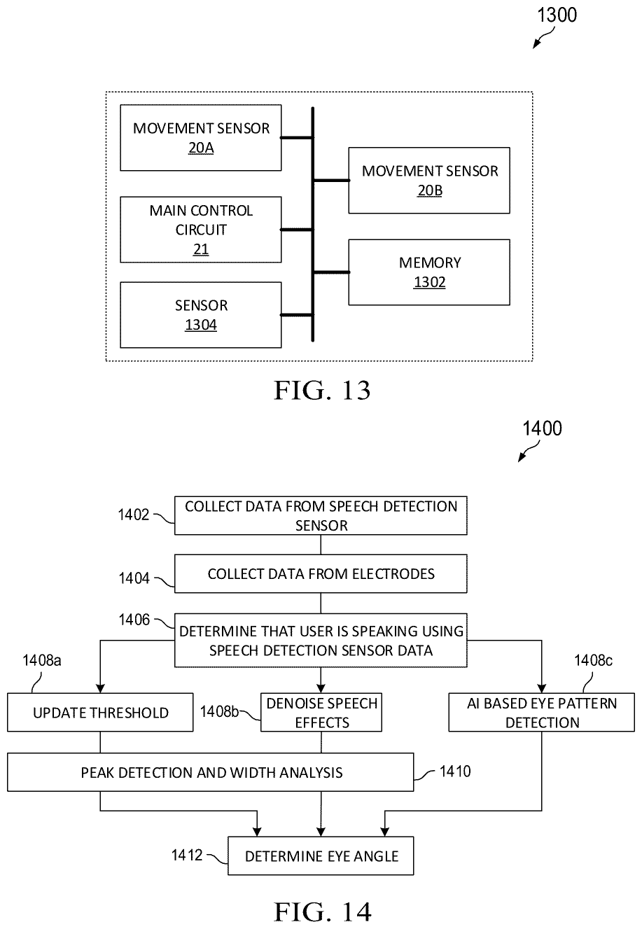

If the user is not speaking, the eye detection process can follow, for example, detection method 150, as outlined in FIG. 9. Conversely, if the main control circuit 21 determines that the user is talking, the eye detection process follows detection method 1400, discussed below in FIG. 14.

FIG. 14 illustrates a flow chart of an embodiment detection method 1400, which may be implemented in device 1300. Detection method 1400 can account for speed-related artifacts. It actively addresses the challenges posed by simultaneous speech, enhancing the accuracy and reliability of eye movement detection in various real-world scenarios.

It is noted that all steps outlined in the flow chart of detection method 1400 are not necessarily required and can be optional. Further, changes to the arrangement of the steps, removal of one or more steps and path connections, and addition of steps and path connections are similarly contemplated.

At step 1402, data is collected from an accelerometer, which may be the sensor 1304 integrated into device 1300. The accelerometer is positioned to detect vibrations associated with the user's head movements and speech. The accelerometer data provides information about the user's physical activity, particularly vocal vibrations, which can interfere with accurate eye movement detection.

During speech activity, the raw data collected from the electrodes is typically affected, which in turn influences, for example, the modulus signal calculated from the collected data. The interference can lead to various issues in eye movement detection, such as false positives where the electrode data erroneously exceeds the threshold value (r_{threshold}) set for identifying eye movements, as the baseline is no longer stable due the speech activity.

Additionally, other aspects of the eye detection method may be compromised due to these speech-related artifacts. Consequently, it is beneficial to implement measures that account for and mitigate the effects of speech-related artifacts in the raw data obtained from the electrodes. Such measures can significantly improve the accuracy and reliability of eye movement detection during periods of vocal activity.

At step 1404, data is collected from electrodes. In embodiments, the electrodes are configured to detect the electrostatic charge variations (Qvar) associated with eye movements. In embodiments, at least two sets of electrodes-one for detecting vertical eye movements and another for horizontal movements—are used. The data collected from the electrodes form the input for eye movement analysis.

At step 1406, in response to determining that the user is speaking by analyzing the accelerometer data, one or more processes may be used to process the raw data collected by the electrodes to determine eye angle. In embodiments, the accelerometer data can detect vibrations associated with speech. The analysis can involve processing the accelerometer signals to identify patterns characteristic of speech-related vibrations. The detection of speech is advantageous because vocal activity can introduce artifacts into the eye movement data, potentially leading to false readings.

It should be noted that using an accelerometer to determine that the user is speaking is non-limiting and other methods to make the determination are contemplated in embodiments. For example, the device 1300 may include microphones, bone conduction sensors, electromyography (EMG) sensors, proximity sensors, infrared sensors, piezoelectric sensor, optical sensor, or strain gauge sensors to determine whether the user is speaking.

In embodiments, determining that the user is speaking can lead to various options to address the speech-related activity. Detection method 1400 includes three possible options that can be used individually or in combination to determine eye angle. These options are detailed in steps 1408a-c. Two or more of the potential processes can be combined to provide more accuracy in determining the eye angle.

At step 1408a, the threshold value (r_{threshold}) for eye movement detection is updated to address the challenge of speech artifacts-provides a new baseline for eye detection when the user is speaking. In embodiments, the new threshold value (r_{threshold}) is calculated by adding an offset threshold (offset_{threshold}) to the original threshold value (r_{threshold}) for non-speech conditions. In embodiments, the offset threshold (offset_{threshold}) is calculated as the average of the modulus signal over a time window, providing a dynamic adjustment that accounts for the signal variability during speech.

For example, immediately after detecting speech-related activity, the average modulus signal is calculated over a succeeding 4-second time window. The offset threshold (offset_{threshold}), set to the average modulus signal over the time window, is added to the original threshold value (r_{threshold}) for non-speech conditions to arrive at the updated threshold value. The updated threshold value is then used for peak detection and width analysis at step 1410.

Adaptive thresholding helps maintain detection sensitivity while reducing false positives due to speech-induced movements. In embodiments, the offset threshold (offset_{threshold}) is calculated and stored in memory 1302.

At step 1408b, a speech effect denoising by filtering is applied to mitigate speech-related artifacts in the eye movement data. In embodiments, denoising includes signal processing techniques designed to isolate and remove artifacts introduced by speech. The denoising algorithm may use information from the speech activity detection sensor and electrode data to identify and filter out speech-related disturbances while preserving genuine eye movement signals. In embodiments, a filter filters out noisy data from the raw data collected by the electrodes. In embodiments, the threshold value (r_{threshold}) for eye movement detection remains the same.

At step 1408c, AI-based eye pattern detection is employed using, for example, machine learning algorithms to distinguish genuine eye movements from speech-induced artifacts. In embodiments, AI-based eye pattern detection includes training a model on a large dataset of eye movements, with and without concurrent speech, enabling the system to recognize and classify eye movement patterns with high accuracy even with speech-related interference. In embodiments, AI-based eye pattern detection includes training a model on a large dataset of eye movements without concurrent speech, enabling the system to recognize and classify eye movement patterns with high accuracy.

In embodiments, the AI-based eye pattern detection employs various machine learning model configurations to determine eye angle during speech activity. In one embodiment, the machine learning model takes a single input—for example, the modulus signal computed from the horizontal and vertical movement channels. In embodiments, the model may have two separate inputs corresponding to the horizontal movement and vertical movement channels directly. The weights of the machine learning model can be determined during a training phase using data collected from the electrodes during, for example, periods of speech activity. This allows the model to learn patterns distinguishing genuine eye movements from speech-related artifacts.

The output of the machine learning model can be configured in several ways depending on the desired granularity of eye angle detection. In some embodiments, the model outputs discrete angle values at 45-degree intervals: 0, 45, 90, 135, 180, 225, 270, and 315 degrees. A simpler configuration may output only the four cardinal directions: up, down, left, and right. As another example, the model can output eight directions: up, down, left, right, up-left, up-right, down-left, and down-right. The choice of output configuration can be tailored to the specific needs of the application. Other combinations of inputs and outputs are also contemplated, allowing for flexibility in the AI-based detection system to balance accuracy, computational efficiency, and application requirements.

At step 1410, peak detection and width analysis are performed on the processed data. This step involves identifying significant peaks in the eye movement signal that correspond to distinct eye movements. In embodiments, the peak detection is based on modulus values that exceed the updated threshold value from data at step 1408a. In embodiments, the peak detection is based on modulus values that exceed the original threshold value after the data is updated to remove the speed-related artifacts at step 1408b. The width analysis examines the duration that the modulus signal exceeds the updated or original threshold values, providing information about the type of eye movement (e.g., quick saccades versus longer fixations). This analysis can help characterize the nature and intent of the user's eye movements.

At step 1412, the eye angle is determined based on the results from previous steps. This determination can include combining the processed electrode data, the results of the peak and width analysis, and any corrections or classifications made by the AI-based detection system. The eye angle calculation provides the final output of the eye tracking system, indicating the direction and extent of the user's gaze.

It is noted that in embodiments where the horizontal and vertical eye movements are not converged into the modulus signal, each channel (i.e., horizontal and vertical channel) is analyzed separately (i.e., updated threshold, de-noising, or AI based) to detect whether the user is looking up or down from the vertical channel data or looking left or right from the horizontal channel data.

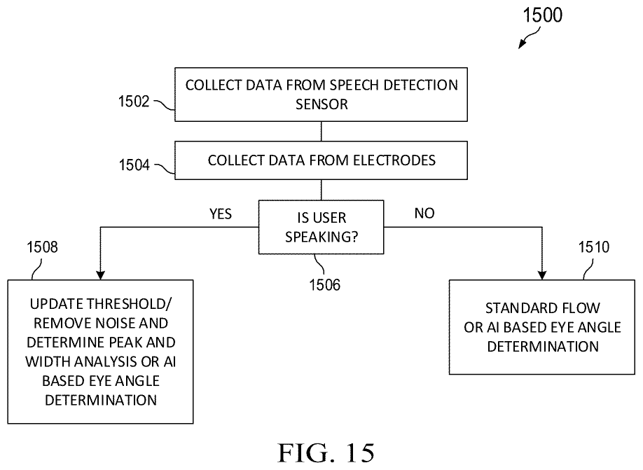

FIG. 15 illustrates a flow chart of an embodiment detection method 1500, which may be implemented in device 1300. It is noted that all steps outlined in the flow chart of detection method 1500 are not necessarily required and can be optional. Further, changes to the arrangement of the steps, removal of one or more steps and path connections, and addition of steps and path connections are similarly contemplated.

At step 1502, data is collected from an accelerometer, which may be the sensor 1304 integrated into device 1300. The accelerometer is positioned to detect vibrations associated with the user's head movements and speech. The accelerometer data provides information about the user's physical activity, particularly vocal vibrations, which can interfere with accurate eye movement detection.

At step 1504, data is collected from electrodes. In embodiments, the electrodes are configured to detect the electrostatic charge variations (Qvar) associated with eye movements. In embodiments, at least two sets of electrodes-one for detecting vertical eye movements and another for horizontal movements—are used. The data collected from the electrodes form the input for eye movement analysis.

At step 1506, the main control circuit 21 determines whether the user is speaking by analyzing the accelerometer data. The accelerometer data can detect vibrations associated with speech. The analysis can involve processing the accelerometer signals to identify patterns characteristic of speech-related vibrations.

At step 1508, in response to detecting speech, the threshold value is updated to address the speech-related artifacts, the data is processed to remove speech-related artifacts, AI/ML techniques, or a combination thereof, are used to determine the eye angle. In embodiments, a weight factor is provided for each potential process to determine the eye angle.

At step 1510, in response to not detecting speech, the threshold value detailed in detection method 150, AI/ML techniques, or a combination thereof are used to determine the eye angle. In embodiments, a weight factor is provided to each process to determine the eye angle.

Although the description has been described in detail, it should be understood that various changes, substitutions, and alterations may be made without departing from the spirit and scope of this disclosure as defined by the appended claims. The same elements are designated with the same reference numbers in the various figures. Moreover, the scope of the disclosure is not intended to be limited to the particular embodiments described herein, as one of ordinary skill in the art will readily appreciate from this disclosure that processes, machines, manufacture, compositions of matter, means, methods, or steps, presently existing or later to be developed, may perform substantially the same function or achieve substantially the same result as the corresponding embodiments described herein. Accordingly, the appended claims are intended to include within their scope such processes, machines, manufacture, compositions of matter, means, methods, or steps.

The specification and drawings are, accordingly, to be regarded simply as an illustration of the disclosure as defined by the appended claims, and are contemplated to cover any and all modifications, variations, combinations, or equivalents that fall within the scope of the present disclosure.

What is claimed is:

1. A circuit for detecting a movement of a first eye and a second eye of a user, the circuit comprising:
a first movement sensor comprising a first electrode and a second electrode aligned along a first movement axis, spaced apart by a first distance, positionable proximate to the first eye, and configured to detect, respectively, a first electrostatic charge variation and a second electrostatic charge variation generated by the movement of the first eye along the first movement axis;
a second movement sensor comprising a third electrode and a fourth electrode aligned along a second movement axis, spaced apart by a second distance, positionable proximate to the second eye, and configured to detect, respectively, a third electrostatic charge variation and a fourth electrostatic charge variation generated by the movement of the second eye along the second movement axis, the second movement axis being transverse to the first movement axis; and
a control circuit coupled to the first movement sensor and the second movement sensor, the control circuit configured to:
acquire a first electrostatic charge variation signal indicative of a difference between the first electrostatic charge variation and the second electrostatic charge variation,
acquire a second electrostatic charge variation signal indicative of a difference between the third electrostatic charge variation and the fourth electrostatic charge variation,
detect, starting from the first electrostatic charge variation signal and the second electrostatic charge variation signal, an event indicative of eye displacement along the first movement axis or the second movement axis, and
determine the movement of the first eye and the second eye based on the event.

2. The circuit of claim 1, further comprising a sensor configured to detect speech-related activity by the user, wherein the control circuit is configured to:
compute a modulus signal from the first electrostatic charge variation signal and the second electrostatic charge variation signal and accounting for the speech-related activity, wherein accounting for the speech-related activity comprises:
removing speech-related artifacts from the first electrostatic charge variation signal and the second electrostatic charge variation signal before computing the modulus signal,
updating a threshold value used to detect the event by adding an offset value to the threshold value in response to detecting the speech-related activity; and
determine an angle at which the first eye and the second eye are directed based on the modulus signal.

3. The circuit of claim 1,
wherein the first movement axis and the second movement axis are orthogonal to each other, or
wherein the first movement axis is parallel to a longitudinal axis of the user's face,
wherein the second movement axis is parallel to, or coincident with, a transverse axis of the user's face,
wherein the longitudinal axis is equidistant from the first eye and the second eye, and
wherein the transverse axis passes through the first eye and the second eye.

4. The circuit of claim 1,
wherein the first electrode and the second electrode extend from sides opposite to each other of the first eye along the first movement axis, and
wherein the third electrode and the fourth electrode extend from sides opposite to each other of the second eye along the second movement axis.

5. The circuit of claim 1,
wherein the first movement sensor further comprises a fifth electrode and a sixth electrode aligned along a first parallel movement axis transverse or orthogonal to the first movement axis, spaced apart by a third distance, positionable proximate to the first eye, and configured to detect, respectively, a fifth electrostatic charge variation and a sixth electrostatic charge variation generated by the movement of the first eye along the first parallel movement axis,
wherein the second movement sensor further comprises a seventh electrode and an eighth electrode aligned along a second parallel movement axis transverse or orthogonal to the second movement axis, spaced apart by a fourth distance, positionable proximate to the second eye, and configured to detect, respectively, a seventh electrostatic charge variation and an eighth electrostatic charge variation generated by the movement of the second eye along the second parallel movement axis, the second parallel movement axis being transverse or orthogonal to the first parallel movement axis,
wherein the control circuit is configured to:
acquire a third electrostatic charge variation signal indicative of a difference between the fifth electrostatic charge variation and the sixth electrostatic charge variation,
acquire a fourth electrostatic charge variation signal indicative of a difference between the seventh electrostatic charge variation and the eighth electrostatic charge variation,
detect, starting from the third electrostatic charge variation signal and the fourth electrostatic charge variation signal, a second event indicative of eye displacement along the first parallel movement axis or the second parallel movement axis, and
determine the movement of the first eye and the second eye based on the second event.

6. The circuit of claim 1, wherein the first electrode, the second electrode, the third electrode, and the fourth electrode are releasably fixed to the user's skin.

7. The circuit of claim 1, wherein the circuit is hosted by a wearable device, the wearable device being an eyewear device, a wearable eyeglass device, a smart wearable eyeglass device, a headset, or an augmented reality headset.

8. A wearable device, comprising:
a frame; and
a circuit for detecting a movement of a first eye and a second eye of a user, the circuit arranged on the frame and comprising:
a first movement sensor comprising a first electrode and a second electrode aligned along a first movement axis, spaced apart by a first distance, positionable proximate to the first eye, and configured to detect, respectively, a first electrostatic charge variation and a second electrostatic charge variation generated by the movement of the first eye along the first movement axis,
a second movement sensor comprising a third electrode and a fourth electrode aligned along a second movement axis, spaced apart by a second distance, positionable proximate to the second eye, and configured to detect, respectively, a third electrostatic charge variation and a fourth electrostatic charge variation generated by the movement of the second eye along the second movement axis, the second movement axis being transverse to the first movement axis, and
a control circuit coupled to the first movement sensor and the second movement sensor, the control circuit configured to:
acquire a first electrostatic charge variation signal indicative of a difference between the first electrostatic charge variation and the second electrostatic charge variation;
acquire a second electrostatic charge variation signal indicative of a difference between the third electrostatic charge variation and the fourth electrostatic charge variation;
detect, starting from the first electrostatic charge variation signal and the second electrostatic charge variation signal, an event indicative of eye displacement along the first movement axis or the second movement axis; and
determine the movement of the first eye and the second eye based on the event.

9. The wearable device of claim 8,
wherein the frame comprises a first support portion and a second support portion coupled to each other and configured to, respectively, face the first eye and the second eye,
wherein the first electrode and the second electrode are fixed to the first support portion and arranged opposite to each other with respect to the first support portion along the first movement axis,
wherein the third electrode and the fourth electrode are fixed to the second support portion and arranged opposite to each other with respect to the second support portion along the second movement axis.

10. The wearable device of claim 8, further comprising a first lens and a second lens, the first lens facing the first eye, and the second lens facing the second eye,
wherein the frame comprises a first support portion and a second support portion coupled to each other and configured to, respectively, face the first eye and the second eye,
wherein the first lens is fixed to, and carried by, the first support portion,
wherein the second lens is fixed to, and carried by, the second support portion,
wherein the first electrode and the second electrode are fixed to, and carried by, the first lens, and
wherein the third electrode and the fourth electrode are fixed to, and carried by, the second lens.

11. The wearable device of claim 8, wherein the wearable device is an eyewear device, a wearable eyeglass device, a smart wearable eyeglass device, a headset, or an augmented reality headset.

12. The wearable device of claim 8,
wherein the first movement axis and the second movement axis are orthogonal to each other, or
wherein the first movement axis is parallel to a longitudinal axis of the user's face, wherein the second movement axis is parallel to, or coincident with, a transverse axis of the user's face, wherein the longitudinal axis is equidistant from the first eye and the second eye, and wherein the transverse axis passes through the first eye and the second eye.

13. The wearable device of claim 8,
wherein the first electrode and the second electrode extend from sides opposite to each other of the first eye along the first movement axis, and
wherein the third electrode and the fourth electrode extend from sides opposite to each other of the second eye along the second movement axis.

14. The wearable device of claim 8,
wherein the first movement sensor further comprises a fifth electrode and a sixth electrode aligned along a first parallel movement axis transverse or orthogonal to the first movement axis, spaced apart by a third distance, positionable proximate to the first eye, and configured to detect, respectively, a fifth electrostatic charge variation and a sixth electrostatic charge variation generated by the movement of the first eye along the first parallel movement axis,
wherein the second movement sensor further comprises a seventh electrode and an eighth electrode aligned along a second parallel movement axis transverse or orthogonal to the second movement axis, spaced apart by a fourth distance, positionable proximate to the second eye, and configured to detect, respectively, a seventh electrostatic charge variation and an eighth electrostatic charge variation generated by the movement of the second eye along the second parallel movement axis, the second parallel movement axis being transverse or orthogonal to the first parallel movement axis,
wherein the control circuit is configured to:
acquire a third electrostatic charge variation signal indicative of a difference between the fifth electrostatic charge variation and the sixth electrostatic charge variation,
acquire a fourth electrostatic charge variation signal indicative of a difference between the seventh electrostatic charge variation and the eighth electrostatic charge variation,
detect, starting from the third electrostatic charge variation signal and the fourth electrostatic charge variation signal, a second event indicative of eye displacement along the first parallel movement axis or the second parallel movement axis, and
determine the movement of the first eye and the second eye based on the second event.

15. A method for detecting a user's eye using a wearable device, the method comprising:
detecting, by a first electrode of a first movement sensor, a first electrostatic charge variation generated by a movement of a first eye along a first movement axis;
detecting, by a second electrode of the first movement sensor, a second electrostatic charge variation generated by the movement of the first eye along the first movement axis, the first electrode and the second electrode aligned along the first movement axis, spaced apart by a first distance, and positionable proximate to the first eye;
detecting, by a third electrode of a second movement sensor, a third electrostatic charge variation generated by a movement of a second eye along a second movement axis;
detecting, by a fourth electrode of the second movement sensor, a fourth electrostatic charge variation generated by the movement of the second eye along the second movement axis, the third electrode and the fourth electrode aligned along the second movement axis, spaced apart by a second distance, and positionable proximate to the second eye, the second movement axis being transverse to the first movement axis;
acquiring, by a control circuit coupled to the first movement sensor and the second movement sensor, a first electrostatic charge variation signal indicative of a difference between the first electrostatic charge variation and the second electrostatic charge variation;
acquiring, by the control circuit, a second electrostatic charge variation signal indicative of a difference between the third electrostatic charge variation and the fourth electrostatic charge variation;
detecting, by the control circuit, an event indicative of eye displacement along the first movement axis or the second movement axis starting from the first electrostatic charge variation signal and the second electrostatic charge variation signal; and
determining, by the control circuit, the movement of the first eye and the second eye based on the event.

16. The method of claim 15, wherein detecting the event comprises verifying whether the first electrostatic charge variation signal and the second electrostatic charge variation signal include a respective peak indicative of eye displacement along the first movement axis and the second movement axis.

17. The method of claim 15, wherein determining, by the control circuit, the movement of the first eye and the second eye based on the event comprises determining the movement using a machine learning model.

18. The method of claim 15, wherein determining the movement of the first eye and the second eye comprises:
mutually correlating the detecting of the eye displacement along the first movement axis and the second movement axis; or
updating a reference angular position of the first eye and the second eye as a function of a preceding reference angular position of the first eye and the second eye detected on the basis of the event, the reference angular position being one of a plurality of predefined reference angular positions.

19. The method of claim 15, further comprising:
verifying a feasibility condition for determining the movement of the first eye and the second eye, the feasibility condition correlated to a presence or an absence of head movements of the user,
wherein acquiring the first electrostatic charge variation signal and the second electrostatic charge variation signal are performed as a function of a verification of the feasibility condition.

20. A non-transitory computer-readable media storing computer instructions that when executed by a processor, causes the processor to:
detect, by a first electrode of a first movement sensor, a first electrostatic charge variation generated by a movement of a first eye along a first movement axis;
detect, by a second electrode of the first movement sensor, a second electrostatic charge variation generated by the movement of the first eye along the first movement axis, the first electrode and the second electrode aligned along the first movement axis, spaced apart by a first distance, and positionable proximate to the first eye;
detect, by a third electrode of a second movement sensor, a third electrostatic charge variation generated by a movement of a second eye along a second movement axis;
detect, by a fourth electrode of the second movement sensor, a fourth electrostatic charge variation generated by the movement of the second eye along the second movement axis, the third electrode and the fourth electrode aligned along the second movement axis, spaced apart by a second distance, and positionable proximate to the second eye, the second movement axis being transverse to the first movement axis;
acquire, by a control circuit coupled to the first movement sensor and the second movement sensor, a first electrostatic charge variation signal indicative of a difference between the first electrostatic charge variation and the second electrostatic charge variation;
acquire, by the control circuit, a second electrostatic charge variation signal indicative of a difference between the third electrostatic charge variation and the fourth electrostatic charge variation;
detect, by the control circuit, an event indicative of eye displacement along the first movement axis or the second movement axis starting from the first electrostatic charge variation signal and the second electrostatic charge variation signal; and
determine, by the control circuit, the movement of the first eye and the second eye based on the event.
